(19) United States
(12) Patent Application Publication
(10) Pub. No.: US 2024/0157115 A1
(43) Pub. Date: May 16, 2024

(54) DEVICE FOR ASSISTANCE BY DIRECT CARDIAC COMPRESSION

(71) Applicants: UNIVERSITE DE LORRAINE, Nancy (FR); VELVET INNOVATIVE TECHNOLOGIES, Messein (FR); ECOLE NATIONALE SUPERIEURE D'ARTS ET METIERS (ENSAM), Paris (FR); INSERM (INSTITUT NATIONAL DE LA SANTE ET DE LA RECHERCHE MEDICALE), Paris (FR); CENTRE HOSPITALIER RÉGIONAL ET UNIVERSITAIRE DE NANCY, Vandoeuvre-les-Nancy (FR)

(72) Inventors: Nguyen TRAN, Seichamps (FR); Antoine CHALON, Nancy (FR); Juan-Pablo MAUREIRA, Ludres (FR); Julien FAVRE, Saint-Etienne (FR); Boris PIOTROWSKI, Chenicourt (FR)

(21) Appl. No.: 18/550,520

(22) PCT Filed: Mar. 17, 2023
(86) PCT No.: PCT/EP2022/056918

§ 371 Date: Sep. 14, 2023

(30) Foreign Application Priority Data
Mar. 17, 2021 (FR) 2102652

(51) Int. Cl. A61M 60/191; A61M 60/289; A61M 60/47; A61M 60/523

(52) U.S. Cl. CPC A61M 60/191; A61M 60/289; A61M 60/47; A61M 60/523

(57) ABSTRACT

An implantable device for cardiac assistance by direct compression, including a base forming a receptacle for receiving the lower part of a heart, a support for fixing the lower part of the heart to the base, at least one finger extending longitudinally between a free upper first end and a curved lower second end inside the base, the finger being mounted on the base via a first pivot connection, a contact interface mounted adjustably and movably on the free end, an actuator for actuating the tilting movement of the finger about the pivot connection, and a control unit for controlling the actuator is disclosed.

The present invention relates to a device for cardiac assistance by direct compression, intended for the treatment of heart failure.

More specifically, the invention relates to an implantable device for pericardial assistance, advantageously fixed to the apex of the heart and capable of exerting a localized pressure at determined places of the outer wall of the heart in order to improve cardiac function. This device can operate synchronously with the patient's native rhythm or according to a rhythm imposed by a stimulation device such as a pacemaker implanted previously or according to a rhythm imposed by a controller.

The device is powered by an energy storage system implanted in a preferential way in the patient's body, and which is preferably designed to be recharged transcutaneously.

The ventricular assistance devices used clinically to assist failing hearts conventionally require direct contact between said devices and the patient's blood.

The risks of thromboembolic events, hemolysis, immune reactions and infections contribute significantly to morbidity and mortality of these devices.

For this reason, direct cardiac compression (DCC), which consists of exerting a pressure on the pericardial surface of the heart, could provide a ventricular support and avoid interactions between the blood and a foreign body.

In 1965, Georges Anstadt developed a pericardial compression device intended for cardiopulmonary resuscitation (CPR). This device, known as the Anstadt cup, is an elliptically shaped cup which fits over the right and left ventricular chambers. It has a semi-rigid outer shell and an inflatable inner diaphragm that delivers compression forces to the heart.

Later, the company Cardio Technologies Inc. developed the CardioSupport® system described in document EP1030701 A1, the subject of which is a ventricular cuff designed to assist a heart to pump blood by applying a uniform pressure over a significant part of an outer ventricular surface of the heart. The ventricular cuff comprises an outer shell, an inflatable inner bladder and a fastening assembly (not shown). The bladder has an opening for communicating with a source of pressurized fluid so that the bladder is inflated cyclically and deflated at a predetermined speed to assist the ventricles of the heart to contract correctly.

Other work described in document U.S. Pat. No. 6,464,655 A1 relates to a device equipped with several fingers for selectively assisting the ventricles, and in particular the left ventricle, of a weak heart to exert a pericardial pressure and to pump blood from one or more sides, in synchrony with the natural systolic contraction of the ventricle. This implantable device comprises a micro-processor to monitor the plurality of fingers which contract once the solenoid is powered electrically and rapidly release the heart when the solenoid is not powered.

However, it has proved necessary to improve the existing devices by proposing a solution in which the pressure exerted on the epicardium by the direct cardiac assistance devices can be very precisely controlled at least in terms of frequency, intensity and location.

For this reason, the invention relates to an implantable device for cardiac assistance by direct compression, characterized in that it includes:
a base forming a receptacle capable of receiving the lower part of a heart,
a support for fixing the lower part of the heart to the base, said support comprising a fixing arm and fasteners at the base, making it possible to adjust the position of said support on said base,
at least one finger extending longitudinally between a free upper first end and a curved lower second end inside the base, said finger being mounted on said base by virtue of a first pivot connection,
a contact interface mounted adjustably and movably on said free end so that said interface can exert an adjustable pressure on a localized area of the epicardium when said finger is moved by a tilting movement about said pivot connection,
means for actuating the tilting movement of said finger about said pivot connection,
a unit for controlling said actuating means.

By “epicardium” is meant the inner layer of the inner side (visceral side) of the pericardium. It constitutes the serous membrane of this side and extends over the entire outer surface of the heart and the proximal part of the great vessels.

Optional characteristics of the invention, additional or replacement, are set out below.

According to certain characteristics, the means for actuating the tilting movement of said at least one finger about said pivot connection can include a connecting rod and a sliding member one of the ends of which of said member is in a second pivot connection with said connecting rod, itself in a third pivot connection with the lower second end of said finger, the other end of said sliding member being connected by means of a rigid or semi-rigid cable to a linear actuator belonging to the control unit.

According to a first embodiment, the device can include six fingers distributed so that the six upper first ends form the vertices of a hexagon, each lower second end of the six fingers being in a pivot connection with a connecting rod, itself in a pivot connection with an end of a sliding member, the other end of said sliding member being connected by means of a rigid or semi-rigid cable to a linear actuator belonging to the control unit, so that the fingers are actuated independently.

According to a second embodiment, the device can include a pair of fingers arranged opposite one another, each lower second end of the fingers being in a pivot connection with a connecting rod, itself in a pivot connection with an end of a sliding member common to each finger, the other end of said common sliding member being connected by means of a single rigid or semi-rigid cable to a linear actuator belonging to the control unit, so that the fingers are actuated identically.

According to other characteristics, the control unit can comprise an electrical storage unit to power the linear actuator(s) and an antenna to allow recharging of said electrical storage unit by the transcutaneous route.

According to yet other characteristics, the control unit can comprise a computer for controlling powering of the linear actuator(s).

According to other characteristics, the support for fixing the lower part of the heart to the base can comprise an area for fastening the apical suture ring.

According to other characteristics, the support for fixing the lower part of the heart to the base can comprise at least one sensor for measuring the intracardiac pressure.

According to other characteristics, the device includes sensors of the flow type capable of being installed on the vessels emerging from the heart, such as the aorta, to allow feedback from said device.

According to other characteristics, each contact interface can be fixed on the free first end of the finger by means of a fastener cooperating with a groove made in said end so as to adjust the position of said interface along said end and thus to vary the location of the pressure.

According to other characteristics, the interface with the epicardium can be equipped with at least one sensor making it possible to measure for example the contact pressure.

According to other characteristics, the computer can be connected to an assembly of sensors internal or external to the cardiac assistance device, in order to adapt the frequency and the intensity of the contact pressure exerted by the first end of the fingers on the epicardium.

Other advantages and features of the invention will become apparent on reading the detailed description of implementations and embodiments which are in no way limitative, and from the following attached drawings:

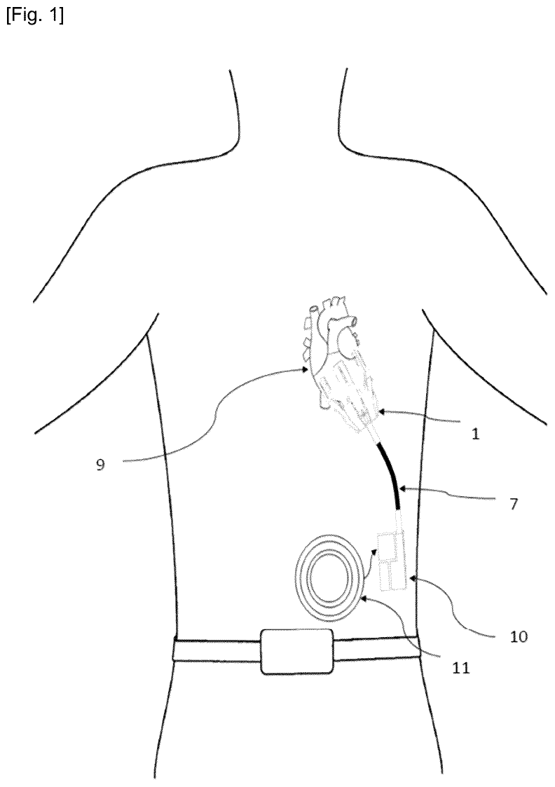

FIG. 1 This figure shows a diagrammatic view of a cardiac assistance device implanted in a patient according to the invention.

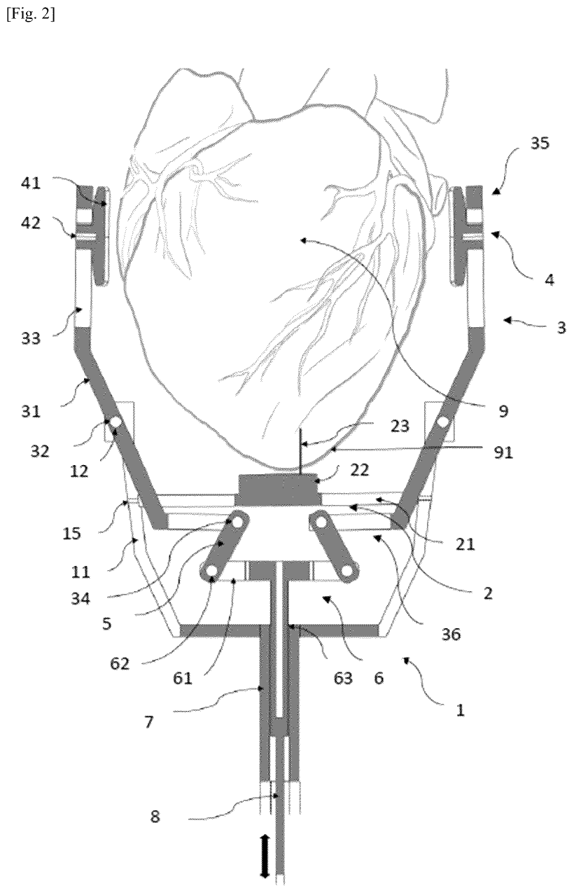

FIG. 2 This figure shows in detail a part of the cardiac assistance device according to an embodiment of the invention.

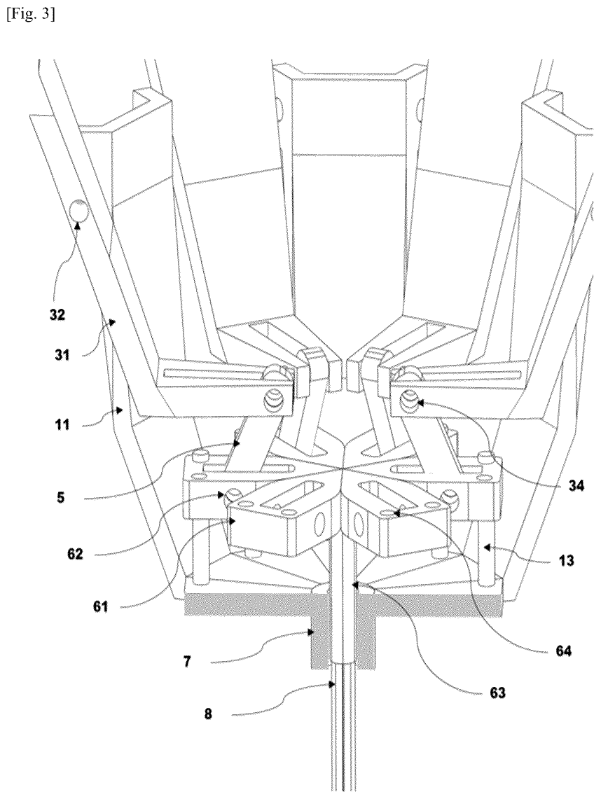

FIG. 3 This figure shows in detail a part of the cardiac assistance device according to another embodiment of the invention.

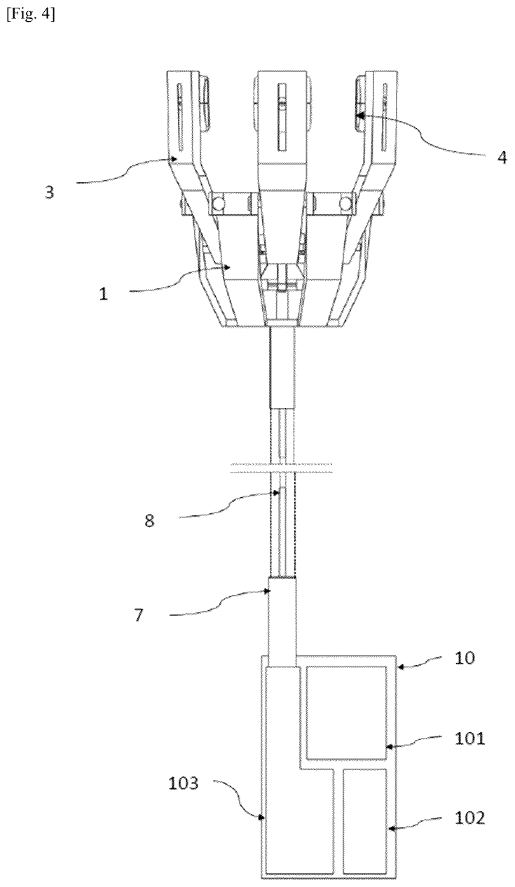

FIG. 4 This figure shows an overall diagram of the cardiac assistance device according to an embodiment of the invention.

As the embodiments described below are in no way limitative, variants of the invention could in particular be considered comprising only a selection of the characteristics described, in isolation from the other characteristics described (even if this selection is isolated within a phrase containing these other characteristics), if this selection of characteristics is sufficient to confer a technical advantage or to differentiate the invention with respect to the state of the prior art. This selection comprises at least one, preferably functional, characteristic without structural details, or with only a part of the structural details if this part alone is sufficient to confer a technical advantage or to differentiate the invention with respect to the state of the prior art.

The device according to the invention is intended to be implanted in patients suffering from terminal heart failure and this is the case in particular where other treatments cannot be implemented.

The invention can also be a substitute for existing treatment solutions if it proves to be more suitable and demonstrates an increased advantage for the patient.

The device according to the invention is intended to be implanted in patients suffering from terminal heart failure and this is the case in particular where other treatments cannot be implemented.

The invention can also be a substitute for existing treatment solutions if it proves to be more suitable and demonstrates an increased advantage for the patient.

The cardiac assistance device according to the invention generates a mechanical deformation of the outer wall of the organ in a manner that is synchronous and compelled by the needs of the failing organ.

The device makes it possible to compensate for the insufficiency of the cardiac pump function without being in direct contact with the patient's circulating blood and without creating a rupture of the skin barrier, a typical source of infection.

The cardiac assistance device according to the invention and as shown in FIGS. 2, 3, 4 includes as a minimum:
a base 1 forming a receptacle capable of receiving the lower part of a heart 9,
a support 2 for fixing the lower part of the heart to the base, said support comprising a fixing arm 21 and fasteners 15 at the base 1, making it possible to adjust the position of said support on said base,
at least one finger 3 extending longitudinally between a free upper first end 35 and a curved lower second end 36 inside the base, said finger being mounted on said base by virtue of a first pivot connection 12,
a contact interface 4 mounted adjustably and movably on said free end so that said interface can exert an adjustable pressure on a localized area of the epicardium when said finger is moved by a tilting movement about said pivot connection,
means 5, 6 for actuating the tilting movement of said finger about said pivot connection,
a unit for controlling 10 said actuating means 5, 6.

Of course, the device according to the invention can include one or more fingers.

The fact that the lower second end 36 of the at least one finger is curved inside the base makes it possible to fit the shape of the heart closely and thus to make the device less bulky.

The first pivot connection 12 of the at least one finger, which is produced for example by means of a hole 32 in the body 31 of said finger, is not arranged at the lower end of said finger, but rather preferably somewhere between the middle of said finger and its lower end.

Thus, by the lever effect, the tilting movement uses less energy.

In all cases, it is the adjustment of the position of the contact interfaces at the level of the ends of the fingers which makes it possible to adjust the contact pressure on the epicardium as well as the precise area to be stimulated.

The device is thus intended to exert a pressure on the heart (systole) and to remain open and distant from the heart in an inactive manner if the actuating means are no longer powered.

This deactivation of the actuating means makes it possible for the heart to dilate freely (diastole) after compression to allow the blood to return naturally and freely towards the cardiac ventricles and the coronary vascular network (arteries and veins).

The external pressure exerted by the myocardium during ventricular systole compresses the cardiac muscle (myocardium) and reduces the blood flow towards the coronary arteries which supply the heart even if the aortic pressure is increased. Thus, more than 70% of the coronary arterial flow occurs during diastole.

According to preferred embodiments shown in FIGS. 2 and 3, the means 5, 6 for actuating the tilting movement of a finger about said pivot connection include a connecting rod 5 and a sliding member 6 one of the ends 61 of which is in a pivot connection 62 with said connecting rod, itself in a pivot connection 34 with the second end 36 of the finger 3.

The other end of the sliding member 6 is then connected by means of at least one rigid or semi-rigid cable 8 to a linear actuator 103 of the control unit 10.

Thus, the cable 8, housed in a sheath 7, makes it possible to offset the motor part, the energy source and the electronic unit at a distance from the thoracic cage, where space is very limited.

The translational movement generated by the linear actuator(s) 103 is transmitted by one or more rigid or semi-rigid cables 8 contained in the sheath 7 up to the part of the device around the heart. The translation of the sliding member 6 in the base 1 is transmitted to the finger 3 via the connecting rod 5 at the level of the pivot connection 62 between the sliding member 6 and the connecting rod 5, then at the level of the pivot connection 34 between the connecting rod 5 and the second end 36 of the finger 3.

This movement causes a tilting movement, i.e. a rotation of the finger about its pivot connection 12 with the base 1, resulting in a concentric reduction of the distance between the ends of the fingers.

According to the first embodiment shown in [FIG. 2], the device includes a pair of fingers arranged opposite one another.

Each lower second end 36 of the two fingers 3 is in a pivot connection 34 with a connecting rod 5 specific to each of the fingers, itself in a pivot connection 62 with an end 61 of a sliding member 6 common to each finger.

The other end of the common sliding member 6 is connected by means of a single rigid or semi-rigid cable 8 to a linear actuator 103 belonging to the control unit 10, so that the fingers are actuated identically.

According to the second embodiment shown in [FIG. 3], the device includes six fingers distributed so that the six upper ends form the vertices of a hexagon.

Each lower second end 36 of the six fingers 3 is in a pivot connection 34 with a connecting rod 5 specific to each finger, itself in a pivot connection 62 with an end 61 of a sliding member 6, specific to each finger. Guiding of the sliding of each member 6 is carried out along at least one axis 13 through an orifice 64 made in the vicinity of the end 61.

The other end of each sliding member 6 is connected by means of a rigid or semi-rigid cable 8 to a linear actuator 103 belonging to the control unit 10, so that the fingers are actuated independently.

One and the same sheath 7 then encloses all six cables 8.

As needed, it is possible to group the sliding members together, to make them interdependent or for them to share one and the same actuating cable or the same linear actuator.

Advantageously and as shown in [FIG. 1], the control unit 10 comprises an electrical storage unit 101 to power the linear actuator 103 and an antenna 11 to allow recharging of said electrical storage unit by the transcutaneous route.

Of course, other configurations for supplying electrical power can be envisaged, with for example the control unit being plugged in externally.

The preferential location for this control unit 10 is the abdomen, with the possibility of recharging the integrated battery by transcutaneous induction.

Preferentially, the support 2 for fixing the lower part of the heart to the base comprises an area for fastening 22 the apical suture ring.

In other words, the device is ideally fixed by suturing between the apex 91 of the heart and the fixing support of the heart 2. This support 2 is mounted on the base 1 by through fasteners 15 at the level of the fixing arms 21.

The assembly formed by the base 1 and the fingers 3 is thus firmly attached to the heart at the level of the apex, and fits its lower part as closely as possible.

The apical suture ring, which is known to a person skilled in the art, is in the present case a device supplementary to the cardiac assistance described in the invention, which acts as an interface between the organ and the device. In fact, one of the key conditions for ensuring the efficacy of the compression of the heart by the invention is to prevent the organ from slipping out as well as an external deformation at the places of interest and not elsewhere.

The feasibility of the fixing of a cardiac assistance device in the apical area of the heart has already been studied in the publication “Chalon A, Favre J, Piotrowski B, Landmann V, Grandmougin D, Maureira J P, Laheurte P, Tran N. Contribution of computational model for assessment of heart tissue local stress caused by suture in LVAD implantation. J Mech Behav Biomed Mater. 2018 June; 82:291-298. doi: 10.1016/j.jmbbm.2018.03.032. Epub 2018 Mar. 28. PMID: 29649657.”

The apical suture ring is generally constituted by two parts.

The outer part is made, according to a first manufacturing method, from medical grade Teflon® (PTFE), or from Dacron® or from another biocompatible and non-resorbable synthetic fabric. According to a second method for manufacturing this outer part, it is possible to use pericardial tissue from another species (bovine, ovine or other) processed in order to be tolerated by the receiving organism. This outer part (outer ring) is fixed to the inner ring by crimping, adhesive bonding and/or suturing so that these two elements remain firmly attached over time.

The inner ring is made from biocompatible material, such as, but not limited to, stainless steel doped with nitrogen, titanium and titanium alloys (Ti—Nb for example) or a polymer (PEEK, UHMWPE or PP for example). This section includes a fixed segment ensuring attachment to the outer ring and a segment of circular flange, allowing solid connection with the cardiac assistance device. A screw or an adjustment lever make it possible to secure this circular flange on the fastening area of the cardiac assistance device according to the invention. The securing and/or locking make it possible to prevent the rotation of the device with respect to the organ.

According to another embodiment, it is possible to arrange a non-circular (for example hexagonal) flange making it possible to lock the elements to one another and limiting the risks of rotation of the suture ring with respect to the cardiac assistance device.

Thus, the outer part is sutured to the apex of the heart and the inner part is locked to the cardiac assistance device to ensure the invention is held in place and good operation thereof.

With respect to the area for fastening 22 the apical ring on the device, it emerges from the support 2, connected through fasteners 15 to the base 1. This fastening area can be cylindrical or have a shape which complements the flange described above so as to prevent any unwanted rotation of the elements with respect to one another. This fastening element is hollow so as to receive tubes and/or pressure sensors. A window in one of the emergences of the base 1 makes it possible for necessary tubes and/or cables to be passed through without disturbing the operation of the invention.

A cannula, tube or needle that is rigid or not 23 emerges from the fastening area 22 and is dimensioned so that its free end opens inside the left ventricle. Directly connected to a sensor in the hollow of the fastening area 22 or by a tube up to a sensor situated at a distance (in particular at the level of the control unit 10), the sensor 23 makes it possible to continuously measure the left ventricular pressure, which will be used by the control unit 10 to adapt the action of the device to the needs of the patient. In one case or another, tube or cables run along the base 1 and follow the route of the sheath 7 to reach the control unit 10.

Advantageously, the device includes sensors of the flow type capable of being installed on the vessels emerging from the heart, such as the aorta, to allow feedback from said device.

Advantageously, the contact interface 4 is fixed on the free first end 35 of the finger 3 by means of a fastener 42 cooperating with a groove 33 made in said end so as to adjust the position of said interface along said end and also to adjust the distance thereof with regard to the epicardium. The movable nature also makes it possible to carry out the replacement of the interface before implantation of the device in the patient.

These adjustable, movable and customized interface elements 4 are thus fixed by virtue of a fastener 42 in the groove 33 of the upper part of the fingers and effect the contact with the epicardium.

This movement generates a compression phenomenon on the outer surface of the heart which causes a pressure increase in the ventricles and promotes the ejection of the blood towards the pulmonary and systemic circulations.

Advantageously, these elements for interface 4 with the epicardium can be equipped with at least one sensor making it possible to measure the contact pressure.

Similarly, the support 2 for fixing the lower part of the heart to the base can comprise at least one sensor 23 for measuring the intracardiac pressure. Thus, pressure sensors can be installed in the support for fixing the heart 2 and connected to cannulas 23 surgically placed in the ventricles.

Consequently, it is possible to determine the amplitude and/or the compressive force of the fingers as a function of the signals collected by this sensor assembly, in particular in order to synchronize the action of the device with the contraction of the heart.

To this end, the control unit 10 includes a computer 102 for controlling powering of the linear actuator 103.

This computer 102 is connected to an assembly of sensors internal or external to the cardiac assistance device, in order to adapt the frequency and the intensity of the contact pressure exerted by the first end of the fingers on the epicardium.

The acquisition and management of these signals is thus carried out in the control unit 10 and more precisely by the computer 102.

Sensors of the flow type can also be installed on vessels emerging from the heart, typically the aorta, to allow feedback from the action of the invention.

Returning to the opposite position allows a release of the assembly of articulations and a return to the initial “open” position. This initial position can be calibrated to exert a minimal pressure on the organ in order to compensate for its pathological dilation.

The items of equipment which make up the invention and which are implanted in the patient are all designed with materials which are recognized scientifically and industrially as biocompatible, or, if necessary, are isolated from the patient by such materials.

The device can be manufactured entirely or in part by 3D printing based on TiNb or PEEK powder.

Mechanical tests on a test bench and on an ex vivo organ under isovolumic conditions were carried out in order to report on the forces and pressures generated by the device as well as the energy required to create these efforts. The results clearly showed a capacity of the device to restore the hemodynamic parameters even on a heart that has completely stopped, namely the intraventricular pressure, the aortic arterial pressure, to those identical to a normal physiological function.

In addition, this made it possible to show that the energy used by the device for this cardiac assistance work remains at a level that is sufficiently low to envisage the miniaturization and total implantation thereof.

The first tests carried out using prototypes of the invention described made it possible to establish a first estimate of the performance of the device.

Firstly, the tests on explanted hearts under isovolumic conditions (no fluid circulation, cardiac cavities were filled with a blood analogue) showed the possibility of achieving intracardiac pressures up to 110 mm Hg, which corresponds to the typical blood pressure in a healthy adult.

Further tests have shown the possibility of going beyond this.

However, these values, which are physiologically much too high, only illustrate the reserve force which the device can make use of and does not come under the intended clinical use thereof.

The device can handle a contraction frequency up to 80 beats per minute without producing a loss of its contractile efficiency. Beyond this, it is possible to reach values of 120 beats per minute, but with premature wearing of the device, a possible deleterious effect on the heart and, finally, a significantly degraded performance.

The time needed to obtain the maximum force of the device was shown to be approximately 100+/−25 ms but can be significantly improved up to 20+/−10 ms.

The energy consumption was estimated at 155 KJ for 24 hours of continuous use at a rhythm of 60 beats per minute. By way of comparison, this is the amount of energy contained in a battery of a laptop computer.

With regard to other conventional treatments, the implantable device for cardiac assistance by direct compression according to the invention has the following advantages:
Technique which conserves the organ
Preservation of the pulsatility of the flow
Reduction of the volume of the cardiac cavity
Increase of the cardiac flow
Absence of direct blood contact
Total implantation of the device (by minithoracotomy) and its supplementary devices in the patient (reduction of infection risks).

In other words, the implantable device for cardiac assistance by direct compression according to the invention is adjusted as close as possible to the morphology of the heart with respect to the healthy pressure areas as well as the delicate areas and the infarcted areas.

It should be noted that the various characteristics, forms, variants and embodiments of the invention can be combined with one another in various combinations, provided that they are not mutually incompatible or exclusive.

1. An implantable device for cardiac assistance by direct compression, comprising:
a base forming a receptacle capable of receiving a lower part of a heart;
a support for fixing the lower part of the heart to said base, said support comprising a fixing arm and fasteners at the base, making it possible to adjust the position of said support on said base;
at least one finger extending longitudinally between a free upper first end and a curved lower second end inside the base, said finger being mounted on said base by virtue of a first pivot connection;
a contact interface mounted adjustably and movably on said free end so that said interface can exert an adjustable pressure on a localized area of the epicardium, when said finger is moved by a tilting movement about said pivot connection;
means for actuating the tilting movement of said finger about said pivot connection; and
a unit for controlling said actuating means.

2. The implantable device for cardiac assistance by direct compression, according to claim 1, characterized in that the means for actuating the tilting movement of said at least one finger about said pivot connection include a connecting rod and a sliding member one of the ends of which is in a second pivot connection with said connecting rod, itself in a third pivot connection with the lower second end of said finger, the other end of the sliding member being connected by means of a rigid or semi-rigid cable to a linear actuator belonging to the control unit.

3. The implantable device for cardiac assistance by direct compression, according to claim 1, characterized in that the device includes six fingers distributed so that the six upper ends form the vertices of a hexagon, each lower second end of the six fingers being in a pivot connection with a connecting rod, itself in a pivot connection with an end of a sliding member, the other end of the sliding member being connected by means of a rigid or semi-rigid cable to a linear actuator belonging to the control unit, so that the fingers are actuated independently.

4. The implantable device for cardiac assistance by direct compression, according to claim 1, characterized in that the device includes a pair of fingers arranged opposite one another, each lower second end of the fingers being in a pivot connection with a connecting rod, itself in a pivot connection with an end of a sliding member common to each finger, the other end of the common sliding member being connected by means of a single rigid or semi-rigid cable to a common linear actuator belonging to the control unit, so that the fingers are actuated identically.

5. The implantable device for cardiac assistance by direct compression, according to claim 4, characterized in that the control unit comprises an implantable electrical storage unit to power the linear actuator(s) and an antenna to allow recharging of said electrical storage unit by the transcutaneous route.

6. The implantable device for cardiac assistance by direct compression, according to claim 2, characterized in that the control unit comprises a computer for controlling powering of the linear actuator(s).

7. The implantable device for cardiac assistance by direct compression, according to claim 1, characterized in that the support for fixing the lower part of the heart to the base comprises an area for fastening the apical suture ring.

8. The implantable device for cardiac assistance by direct compression, according to claim 1, characterized in that the support for fixing the lower part of the heart to the base comprises at least one sensor for measuring the intracardiac pressure.

9. The implantable device for cardiac assistance by direct compression, according to claim 1, characterized in that the device includes sensors of the flow type capable of being installed on the vessels emerging from the heart, such as the aorta, to allow feedback from said device.

10. The implantable device for cardiac assistance by direct compression, according to claim 1, characterized in that the contact interface is fixed on the free first end of its finger by means of a fastener cooperating with a groove made in said end, so as to adjust the position of said interface along said end.

11. The implantable device for cardiac assistance by direct compression, according to claim 1, characterized in that the interface for contact of each finger with the epicardium is equipped with at least one sensor making it possible for example to measure the contact pressure.

12. The implantable device for cardiac assistance by direct compression, according to claim 6, characterized in that the computer is connected to an assembly of sensors internal or external to the cardiac assistance device, in order to adapt the frequency and the intensity of the contact pressure exerted by the first end of the fingers on the epicardium.
